(19) United States
(12) Patent Application Publication
(10) Pub. No.: US 2024/0197239 A1
(43) Pub. Date: Jun. 20, 2024

(54) METHOD OF ENGAGING OLDER ADULTS THROUGH MEANINGFUL ACTIVITIES

(71) Applicant: Maria's Place, LLC., Eagle, CO (US)

(72) Inventors: Nichole Bontrager, Gypsum, CO (US); Maria Brady; Ryan Iguchi, Torrance, CA (US)

(73) Assignee: Maria's Place, Eagle, CO (US)

(21) Appl. No.: 18/066,705

(22) Filed: Dec. 15, 2022

(51) Int. Cl. A61B 5/00; G16H 20/70

(52) U.S. Cl. CPC A61B 5/4088; G16H 20/70

(57) ABSTRACT

The invention relates to providing a system for maintaining and stimulating brain activity and for providing stimulation for various mental and motor functions for at least one individual having at least one cognitive impairment or physical impairment the system comprising a plurality of games having: at least one game relating to words, at least one game relating to visual and spatial and orientation, at least one game relating to numbers, and at least one game relating to shapes and colors, at least one game relate d to physical activity.

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

Trademarks used in the disclosure of the invention, and the applicants, make no claim to any trademarks referenced.

### CROSS-REFERENCE TO RELATED APPLICATIONS

### Background of the Invention

### 1) Field of the Invention

The invention relates to the field of recreation therapy for older adults and provides a system for maintaining and stimulating brain activity and for providing stimulation for various mental and motor functions for at least one individual.

### 2) Description of Related Art

Currently the state of the art includes a various methods wherein an individual is incentivized to increase both mental and physical activity. However, science has not found a cure for cognitive impairments like Alzheimer's and dementia where research has shown that the brain is capable of making new connections to replace ones that have been lost at the early stages of these types of impairments.

This ability of the brain is called neuroplasticity. Recent findings suggest that cognitive training can help create and sustain these new connections.

After an individual has been diagnosed or prior to diagnosing cognitive impairment, there is a tendency of patients and caregivers to focus attention solely on the functions that have been lost.

This leaves the intact cognitive functions unrecognized and unreinforced. While the gradual decline of cognitive functioning cannot be stopped entirely, some cognitive training and stimulation may play a significant role in slowing down the physiological progress of these diseases. Such training may maximize an individual's ability to function for an extended period of time.

In particular there is a need for a solution to at least one of the aforementioned problems. For instance, there is a need for a system that provides clear and easy to implement by the caregiver kit of activities and social interactions which can be used by older adults and their caregivers.

### BRIEF SUMMARY OF THE INVENTION

The invention relates to the field of providing a system for maintaining and stimulating brain activity and for providing stimulation for various mental and motor functions for at least one individual having at least one cognitive impairment or physical impairment the system comprising a plurality of games having: at least one game relating to words, at least one game relating to visual and spatial and orientation, at least one game relating to numbers, and at least one game relating to shapes and colors, at least one game related to physical activity. The present invention further provides instructions to play the games and means for assessing the results of the games played according to pre-determined criteria.

In a first implementation of the invention, is a computer application that provides activities and social interactions for older adults and their caregivers which is done through an interactive game and clubhouse style chat rooms.

The instant invention solves the problem of lack of accessibility to and usability of suitable adult activities for seniors, caregivers, and people who have dementia.

The computer application provides a simple, easy-to-use computer application with two therapeutic recreation features. One is an interactive brain game, and the other is an audio chat room. The app is catered towards older adults who experience age related deterioration in vision, auditory, touch, and cognition. The computer application has high-visibility design and large fonts to play brain games alone or interact with other app users through different conversations in the audio chat rooms. Users can connect with each other, follow each other, and receive audio notifications when their peers are online. The computer application offers therapeutic recreation in seniors' homes, building feelings of social connection while relieving feelings of isolation or loneliness.

The computer application has two main therapeutic recreation-based features. One is a moderated social audio chat room where users can join and interact with other users and the chat room host, an employee or caregiver of the organization suppling the computer application. The other is an interactive word search brain game that can be played at any time.

Alternative embodiments can include a box or kit containing activities, content and gift items designed to address the patient, client or user needs.

Alternative embodiments of the invention solve the above-mentioned problems by providing a user engagement system that is created and/or organized for a specific patient, client or user.

Alternative embodiments of the invention are directed to a user engagement system. The user engagement system is customized to meet the needs of the patient, client or user.

Alternative embodiments of the invention are directed to a non-transitory computer readable medium having a computer program thereon. The computer program instructs at least one processing element to perform the steps discussed herein.

This summary is provided to introduce a selection of concepts in a simplified form that are further described below in the detailed description. This summary is not intended to identify key features or essential features of the claimed subject matter, nor is it intended to be used to limit the scope of the claimed subject matter. Other aspects and advantages of the invention will be apparent from the following detailed description of the embodiments and the accompanying drawing figures.

These and other objects, features, and advantages of the present invention will become more readily apparent from the attached drawings and the detailed description of the preferred embodiments, which follow.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. When reference is made to a reference numeral without specification to an existing sub-label, it is intended to refer to all such multiple similar components.

FIG. 1, there is shown a simple system diagram of the instant invention;

FIG. 2 there is shown a patient or individual using a smart device or computer and application to access website and displaying the activities of the instant invention;

FIG. 3 there is shown the activity box 250 of the instant invention;

FIG. 4 there is shown a front view of an All-In-One Weaving Kit;

FIG. 4A shows a side view of the All-In-One Weaving Kit;

FIG. 5 there is shown a wood puzzle for people with dementia;

FIG. 6 is a table settings puzzle for people with dementia;

FIG. 7 which shows a season, reminiscing target game for people with dementia; and

FIG. 8 is a shape, color, and numbers floor target game for people with dementia;

and

FIG. 9 shows a typical activity box.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION

While various aspects and features of certain embodiments have been summarized above, the following detailed description illustrates a few exemplary embodiments in further detail to enable one skilled in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art however that other embodiments of the present invention may be practiced without some of these specific details. Several embodiments are described herein, and while various features are ascribed to different embodiments, it should be appreciated that the features described with respect to one embodiment may be incorporated with other embodiments as well. By the same token however, no single feature or features of any described embodiment should be considered essential to every embodiment of the invention, as other embodiments of the invention may omit such features.

In this application the use of the singular includes the plural unless specifically stated otherwise and use of the terms “and” and “or” is equivalent to “and/or,” also referred to as “non-exclusive or” unless otherwise indicated. Moreover, the use of the term “including,” as well as other forms, such as “includes” and “included,” should be considered non-exclusive. Also, terms such as “element” or “component” encompass both elements and components including one unit and elements and components that include more than one unit, unless specifically stated otherwise.

Lastly, the terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

As this invention is susceptible to embodiments of many different forms, it is intended that the present disclosure be considered as an example of the principles of the invention and not intended to limit the invention to the specific embodiments shown and described.

The terms individual, patient, client or user are used interchangeably to mean an individual who utilizes the instant invention.

The term caregiver as used in the specification is meant to mean a person who works with an individual patient, client or user who uses the instant invention.

The term app, application, computer program are used interchangeably to mean a computer program that uses a computer, smart device, smart tablet or smart phone and is utilized by the instant invention.

Prior to a discussion of the preferred embodiment of the invention, it should be understood that while the features and advantages of the invention are illustrated in terms of an of a computer application or a manual system for providing activities to a patient, client or user that the applicant also envisions various alternatives to provide the instant invention such as but not limited to computer application, an application on a smart device such as a smart phone or tablet and manual methods of delivery of the activity methods and games such as mail, courier and other deliver options. Where manual deliver is used the container can be any suitable container such as a box, bag or envelope.

The instant invention provides activities aim to increase or maintain the quality of life and independence in older adults through focusing on the social, emotional, cognitive and physical domains of life. They are adaptable to different ability levels and are made specifically for older adults. It provides instructions on how to do the activities as a caregiver caring for people with different ability levels in a one-on-one or, where applicable, in group settings. Our activities are intergenerational, and are encouraged to be done with family members, friends, or younger people in order to foster social connections.

The activities of the instant invention consider different ability levels and the step-by-step instructional aspect with this is unique. The vast number of activities on the website gives people an instant resource, and they can also have the activities in their hands as physical products in the form of activity boxes. The instant invention provides a fully customizable activity box for organizations who serve older adults. This does not exist in the marketplace and the people with dementia suffer because of the lack of activity suitable for them. The physical products of the instant invention for people with dementia and people with various physical limitations are unique in their design and specifically catered toward ease of use while still providing a manageable challenge for the user. The instant invention can alternatively be used for people with dementia or various other disabilities.

The instant invention provides activities to stimulate the cognitive, physical, social and emotional life domains in older adults. It does this through multiple interventions.

The website is a place where users can find a variety of activities in the areas of crafts, games, brain teasers, physical exercise, relaxation and reflection, reminiscence, and sensory activities.

The instant invention application can send activity kits to older adults. Each kit includes a craft with craft supplies, a recipe, a reminiscing story, brain teasers and a guided physical exercise routine.

The instant invention makes caregiver appreciation/wellness boxes which can be used by the caregiver when working with patients with dementia or to assist in their overall training.

The instant invention dementia specific physical products are designed for different levels of dementia—easy grip, visually enticing, creative, and tactile with an emphasis on problem solving and decision making.

The instant invention application aims to provide activities and social interactions among older adults—this is done through an interactive game and clubhouse style chat rooms.

The instant invention provides a system with multiple uses. The activities can also be used by people aging independently at home. They can be used as a tool for family caregivers to engage in activities with the people they are caring for. They can be used in residential living communities, senior day centers, and retirement communities. In-home care agencies can use the activities with their older adult clients. Area Agencies on Aging can send the boxes to their homebound older adult clients. Insurance companies can use the boxes with their Medicare enrollees. Organizations who serve older adults in any capacity can use the boxes with their clients.

Further, the instant invention provides easy access to suitable activities for older adults. The kits provide the preparation work and planning, sourcing and instructing so the user can simply do the activity. The invention saves time for everyone in the care community and provides the education to caregivers on recreation therapy for older adults.

The instant invention is a device which provides recreation therapy for seniors, caregivers and people who have dementia to solve the problem of lack of easy access to activities built specifically for older adults. The instant invention can also address the lack of accessibility to and usability of suitable adult activities for seniors, caregivers, and people who have dementia.

As documented in https://www.cdc.gov/physicalactivity/inactivity-among-adults-50plus/index.html by age 75, about one in three men and one in two women engage in no physical activity, causing many negative health effects (CDC).

A sedentary lifestyle has been linked to an increased chance of all-cause mortality in older adults” (Park et al., 2020) https://www.ncbi.nlm.nib.gov/pmc/articles/PMC7700832/,

https://www.cdc.gov/aging/publications/features/lonely-older-adults.html

From the National Academies of Sciences, Engineering, and Medicine (NASEM) points out that more than one-third of adults aged 45 and older feel lonely, and nearly one-fourth of adults aged 65 and older are considered to be socially isolated. Older adults are at increased risk for loneliness and social isolation because they are more likely to face factors such as living alone, the loss of family or friends, chronic illness, and hearing loss.

Social isolation was associated with about a 50% percent increased risk of dementia. 1

https://www.ncbi.nlm.nih.gov/pmc/articles/PMC8691185/#bib9

Unsurprising given that more than 90% of U.S. adults have never received caregiver training (Burgdorf et al., 2019).

In addition, untrained caregivers are at high risk of experiencing excess burden, chronic stress, and depression (National Academies of Sciences, Engineering, and Medicine, 2016; National Alliance for Caregiving. 2020), and their lack of training may also result in increased service utilization by the care recipient.

Over 25% of seniors in their 60s say they are not confident that their communities will have the resources and services they need to lead a healthy and independent life over the next five to 10 years (RHI Hub)

Research published in the American Journal of Epidemiology found that stimulating activity, both socially or mentally oriented, can preserve cognitive function in the elderly and may reduce the risk of developing dementia. (https://www.nursenextdoor.com/blog/6-ways-recreational-therapy-helps-seniors/)

Physical: Research from 2018 Trusted Source shows that recreational therapy involving physical activity may decrease symptoms of depression.

Brain Health: Several studies have established a link between participation in self-initiated, cognitively stimulating activities and brain health (Mintzer et al., 2019).

Community: Individuals with strong community links have been associated with mental well-being and better brain health compared with adults who don't (Mintzer et al., 2019). https://www.atra-online.com/page/AboutRecTherapy.

These references make a clear case for Recreation therapy as the solution to postponing the effects of dementia in elderly patients.

The instant invention comprises the following alternative components:
a. a website;
b. a computer application;
c. an activity box; and
d. various physical products.

The instant invention provides the patient activities aim to increase or maintain the quality of life and independence in older adults through focusing on the social, emotional, cognitive and physical domains of life. The instant invention is adaptable to different ability levels and are made specifically for older adults. The instant invention provides instructions on how to do the activities so that a caregiver caring for people with different ability levels in a one-on-one or, where applicable, in group settings. The activities are intergenerational, and are encouraged to be done with family members, friends, or younger people in order to foster social connections.

The instant invention provides for a website which is a place where users can find activities for caregivers and older adults for physical, social, emotional and cognitive wellbeing. The website content is designed to provide a number of alternatives for the caregivers and patients and has over 700 different activities that can be accessed and used by the caregivers and patients. The instant invention provides directions for caregivers on how to adapt the activities to different ability levels and also in different scenarios like in a one-on-one or group setting if applicable. Each activity box contains activities that cater to the physical, cognitive, social and emotional life domains of the user. The activity boxes are fully customizable for organizations so that they can add their own information and items along with our activities. This ensures that each organization is meeting their goals and objectives and reaching their clients in a friendly and engaging way. The physical products we have invented for dementia and people with various physical limitations are unique in their design and specifically catered toward ease of use while still providing a manageable challenge for the user.

The instant invention provides all parties with a system that is easy to use, visually enticing, ability to be modified for different ability levels, designed specifically for older adults, the sheer size of the resource (# of activities available and variety), the caregiver specific directions that come with activities. The games and activities use big fonts and simplify all designs to increase ease of use.

The biggest thing that makes instant invention unique for corporate clients is that the kits are fully customized activity boxes specific for an organization and the older adult clients they support.

The instant invention provides convenience because it is a saves time. All the work into planning and creating activities are already done so that the users can just grab and go. The instant invention teams members are experts in the activity and recreation therapy department, so we are a trusted, reliable source of activities for caregivers and their loved ones.

The physical activities include follow along yoga videos and seated exercise routines, arts and crafts to use fine motor skills, recipes for cooking and sensory activities to stimulate the senses of touch, hearing, sight, smell and taste.

Activities can be used by other demographics and repurposed for children, people of any age with cognitive disabilities. Which can include more physical products, specifically designed for older adults or people with physical impediments, wellness boxes for any demographic, recreation therapy and dementia courses, volunteering at residential care communities, courses and seasonal activity boxes, holiday specific boxes, birthday boxes.

To address the social and emotional domains of life the website provides activities to be done one-on-one or in group settings. The website has advice on how to adapt activities to different ability levels and situations. The website has a journal and the caregiver stories section to give real-life life stories and experiences of caregivers and activity professionals working with older adults. The website members are offer a monthly social gathering via zoom where members come together with staff to connect. Activities on reflection, reminiscing, meditations, and scenic videos cater to the emotional wellbeing of our users.

On the cognitive activity side of the website, users can find quizzes, word games, trivia, memory games, coloring, painting, art, crafts, seasonal activities and informative articles.

Each activity has directions for caregivers on how to adapt the activity for dementia, group settings, and one-on-one. This makes the activities suitable for people working in the senior care sector.

The search function filters by the type of activity, ability level, or specific topics. The site is designed for optimal ease of use through large, legible fonts, simple navigation, and appealing visuals. Written activities on the site are all downloadable and printable.

The instant invention provides for activities and social interactions among older adults which is done through an interactive game and clubhouse style chat rooms.

The instant invention uses a simple, easy-to-use iOS/Android application with two therapeutic recreation features. One is an interactive brain game, and the other is an audio chat room. The application is designed to accommodate the needs of older adults who experience age related deterioration in vision, auditory, touch, and cognition. The application has high-visibility design and large fonts to play brain games alone or interact with other application users through different conversations in the audio chat rooms. Users can connect with each other, follow each other, and receive audio notifications when their peers are online. Our application offers therapeutic recreation in seniors' homes, building feelings of social connection while relieving feelings of isolation or loneliness.

The instant invention application has two main therapeutic recreation-based features. One is a moderated social audio chat room where users can join and interact with other users and the chat room host, an employee at the organization running the instant invention. The other is an interactive word search brain game that can be played at any time. The intersection between our human centered design and therapeutic recreation provides the user with a fulfilling and unique experience that stimulates their cognitive environment.

The instant invention application is adaptive and is designed with age-related changes in mind. This application provides high contrast between text and background to enhance legibility. The font size on the application will be at least 12 pt, while the fonts on the printed directions will be 18 pt or larger. Simplicity is the cornerstone of the design and is key in avoiding visual clutter and creating a central visual field. To accommodate individuals with audio or visual impairments, all instructions are narrated in English and volume controlled by the user's preference. The user can choose a male or female narrator, and the volume control is highlighted for ease of use. The verbal information has a predictable and calming linguistic pattern that promotes a sense of security. The button size on the application has a large “tap” zone with increased button sizes that make a click sound when tapped correctly which provides an audio confirmation for the user. The application has a help icon on each screen where a user can tap and watch a how-to video that explains the contents of that screen. If further assistance is needed, there is an option to contact a live person which is located alongside the help video icon.

The application is designed with features to increase older adults' ability to navigate and utilize the application independently. Each screen has a maximum of two options which is a novel design concept and reduces the screen complexity for those with cognitive issues and therefore makes the use more enjoyable and reduces the frustration level of navigating the screens. The limited choices simplify the application and aim to lower the feeling of being overwhelmed during navigation. The application contains familiar and repetitive language to increase technological comprehension and learnability.

The instant invention also provides for the delivery of the system manually by providing an activity box so that those patients that do not have access to a smart device or computer can receive activities and benefit from the instant invention.

The creation of the activity box addresses those individuals that do not have access to internet. When looking at the elderly population this can be a large percentage of the individuals that can benefit from the instant invention. The instant invention creates a container with the select activities into a container or physical box and ships them to the patient or individual. Each box focuses on quality of life through addressing the needs of the recipient's social, emotional, physical and cognitive life domains.

The standard monthly activity box includes an activities booklet of the month. The booklet includes 5 brain teasers, 1 craft activity, an exercise routine, a recipe and story, planning (calendar and to-do area) and journaling prompts. The box also includes the craft supplies needed to complete the crafts in the booklet. Each week the recipient is provided an activity challenge for the recipient and at the end of the month, recipients and instant invention team members have an interactive activity social via Zoom to review results and provide coaching.

Family members of older adults are the usual purchaser of the boxes as a gift to keep their loved ones active and engaged. The boxes are an excellent vessel for socializing—users can do the activities with other people and they can also join their fellow community of users of the instant invention for interaction.

A unique aspect of this activity box is that they can be completely customize the boxes for organizations who serve older adults. The instant invention can be co-branded so that the boxes are customized with specific activities, content and gift items go into the boxes based on each organization's specific goals and initiatives. The boxes can be customized to be dementia-friendly.

These boxes can also be used by insurance companies as a:
a. Wellness program for Medicare subscribers.
b. Welcome for new Medicare enrollees.
c. Customer appreciation gift.
d. Marketing vessel to both supply activities and create awareness of the other benefits available to subscribers.

The activity boxes can also be used by Area Agencies on Aging (AAA) as an activity program for their older adult clients. AAA's can also provide a Caregiver Appreciation Box to their caregiver clients. These boxes include activities made for caregivers to induce wellness and engagement.

The boxes are also used by In Home Care Agencies as a welcome box and activity program for their paid caregivers to bring into the homes of the older adults they serve.

Other organizations who use these boxes for their older adult clients include senior care facilities like nursing homes, assisted living, independent living, senior day centers and retirement communities. Any other organization who serves older adults is an ideal purchaser and distributor of the activity boxes.

The instant invention can have an activity box embodiment which is made to meet the needs of a specific organization and their activity. The contents of the activity box can be anything that the organization wants delivered to the user and it can have but not limited to items with the organization logo, an activity booklet, welcome information and gift items. The items with the organization logo, activity booklet, welcome information and gift items are placed in box for delivery to the user. The activity box can also have the organization logo on it.

The instant invention also provides patients and individuals with dementia specific products, which are designed for different levels of dementia, with an emphasis on problem solving and decision making. Some general features include being visually enticing, creative, easy grip and tactile.

These include:
All-In-One Weaving Kit;
Wood Puzzle for dementia;
Table Settings Puzzle for dementia;
Seasons, Reminiscing target game for dementia; and
Shape, color, and numbers floor target game for dementia

The system of the instant invention is targeted at maintaining and stimulating brain activity and for providing stimulation for various mental and motor functions for at least one individual having at least one cognitive impairment or physical impairment. The system comprising a plurality of games having at least one game relating to words, at least one game relating to visual and spatial and orientation, at least one game relating to numbers, and at least one game relating to shapes and colors, at least one game relate d to physical activity.

The instant invention can also be assembled into a kit where of all the items are provided for used of the games and activities for a typical patient or individual simple and the kit is easy for the user to use because all the parts necessary for the patient or user are contained in one easy to use container or box which makes the use easy for a patient and caregiver.

The instant invention can further be summarized as an interactive game kit for use by a person with dementia comprising:
a. a smart device;
b. a cloud application on a cloud server;
c. an application on the smart device;
d. an activity box;
e. the activity box containing at least one game;
f. at least one player having dementia wherein the at least one player having dementia receives the activity box and the at least one player having dementia using the application on the smart device access a website and the website communicating with the application on the smart device and the application on the smart device verifies the smart device is a sanction device using the IP address of the smart device and if the IP address is on a sanction IP list on the cloud application on a cloud server then the application on the smart device is allowed access to the website; and
g. the website communicates with the application on the smart device and displays instructions for the at least one game.

The interactive game can also have the following alternative embodiments wherein said smart device is selected from the group consisting of a computer, a smart device, a smart tablet and a smart phone

The interactive game kit can also have the following alternative embodiments wherein the at least one game s selected from the group consisting of an all-in-one weaving kit, a wood puzzle for dementia, a table settings puzzle for dementia, a season, reminiscing target game for dementia, and a shape, color, and numbers floor target game for dementia

The instant invention can also have the following alternative embodiment which is a kit comprised of an activity box for use by a person with dementia comprising:
a. the activity box containing at least one game; and
b. the activity box containing an instruction sheet having instructions for the at least one game in the activity box.

The kit of the alternate embodiment wherein the at least one game s selected from the group consisting of an all-in-one weaving kit, a wood puzzle for dementia, a table settings puzzle for dementia, a season, reminiscing target game for dementia, and a shape, color, and numbers floor target game for dementia

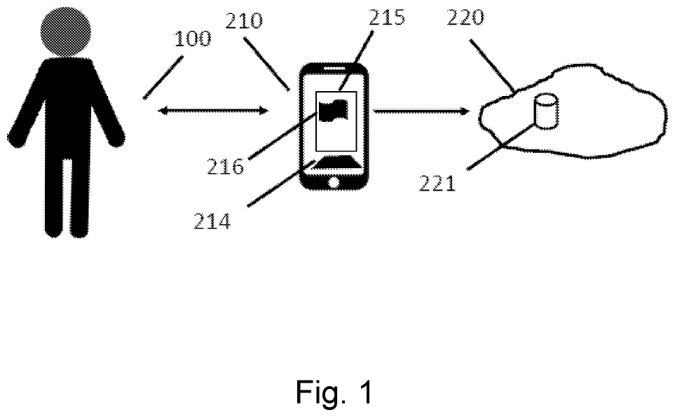

Referring now to the drawings FIGS. 1-16, and more particularly to FIG. 1, there is shown a simple system diagram of the instant invention where the patient or individual 100 uses a smart device 210 or computer and application 214 to access website 215 and displaying the activities 216 of the instant invention. The activities 216 are stored in the cloud database 221 which is part of the cloud 220.

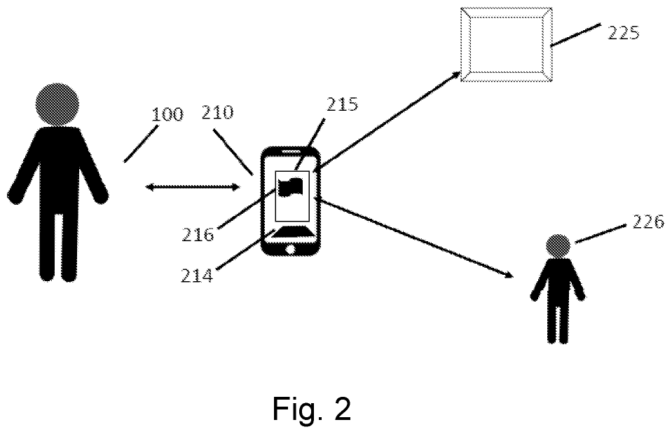

Referring to FIG. 2, there is shown a patient or individual 100 using a smart device 210 or computer and application 214 to access website 215 and displaying the activities 216 of the instant invention. The instant invention application has two main therapeutic recreation-based features. One is a moderated social audio chat room 225 where users can join and interact with other users and the chat room host 226, an employee at the organization running the instant invention. The activities 216 can be an interactive word search brain game that can be played at any time. The intersection between the human centered design and therapeutic recreation provides the user with a fulfilling and unique experience that stimulates their cognitive environment.

The system is protected from unauthorized used by the incorporation of a security system which limits access to the website to only those IP address that are on a sanctioned list of allowed users. First the user 100 logs into an application on a first smart device and the application sends the smart device internet protocol address to the cloud application. The cloud application has a sanction list of user internet protocol IP addresses to ensure that the user is the sanctioned user. This ensures that the user 100 identity and privacy is maintained and that a person who is not authorized or intended to participate in a specific session is prevented for entering the session.

The user 100 using the application 214 upon initial log in registers their smart device 210 and the application captures the smart device 210 IP address and stored it in a sanctioned IP address list located on the cloud database 210. Therefore, during m=normal utilization the user logs into the website 215 and the application 214 checks the IP address and with the sanctioned list of IP addresses stored in the sanctioned IP address list located on the cloud database 210. If there is a match then the application 214 is allowed to access the website 215.

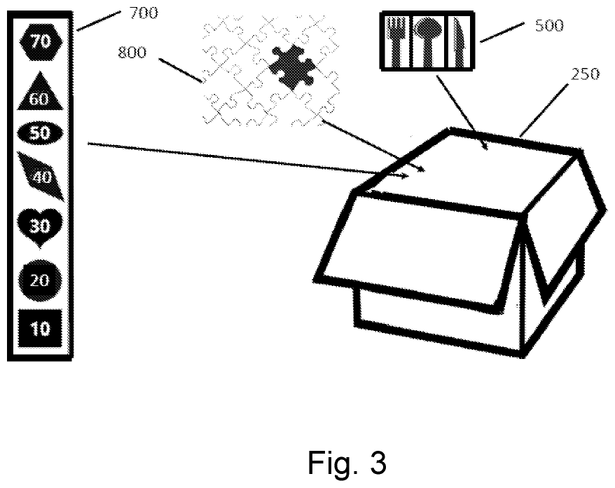

Referring to FIG. 3 there is shown the activity box 250 of the instant invention wherein the box 250 has puzzle or activity type loaded into the box and in this example the puzzle 800, Table Settings Puzzle for dementia 700 and Shape, color, and numbers floor target game for dementia 700 are placed in the box for distribution. The activity box 250 can be coupled with the application 214 or have an instruction sheet or manual to assist the patient or individual 100 to use the puzzle or activity type. The patient or individual 100 uses a smart device 210 or computer and application 214 to access website 215 and displaying the activities 216 of the instant invention. The instant invention application 214 then determines the activity box received by the patient or individual 100 or the patient or individual 100 can select the appropriate activity box. The patient or individual 100 is then guided by the application 214 to play the various games or activities in the box. There are help features and instructions associated with each game or activity in the box. The activities 216 can be an interactive word search brain game that can be played at any time. The intersection between the human centered design and therapeutic recreation provides the user with a fulfilling and unique experience that stimulates their cognitive environment.

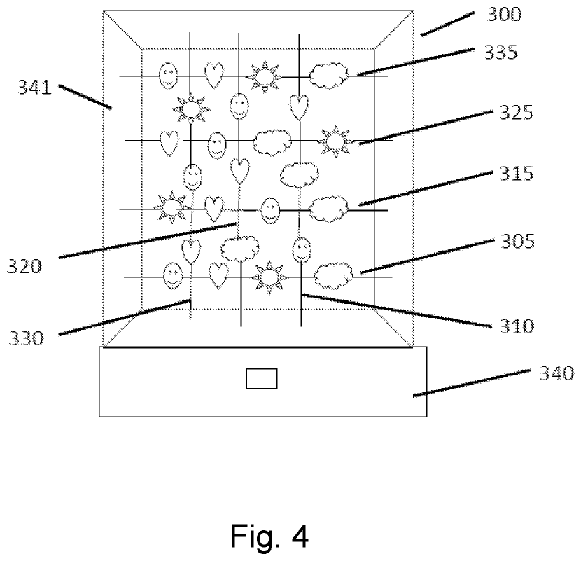

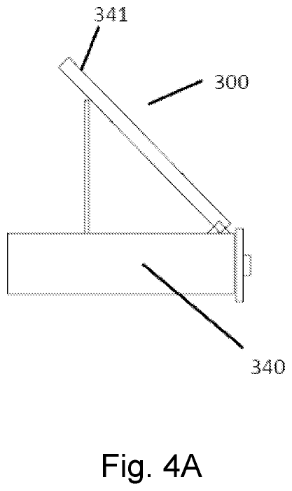

Referring to FIG. 4 there is shown a front view of an All-In-One Weaving Kit 300 having horizontal weaving members 335, 325, 315 and 305 and vertical weaving members 310, 320 and 330. FIG. 4A shows a side view of the All-In-One Weaving Kit 300 with collapsible wood frame 341 attached to the front-top side of the drawer 340.

The All-In-One Weaving Kit 300 is a non-competitive activity that consists of a drawer, with an easy-grip drawer pull. A collapsible wood frame 341 is attached to the front-top side of the drawer 340. The frame has a number of permanent strings running top to bottom, spaced one inch apart. The wood frame can lie flat on the box or be adjusted at an angle to face the person doing the weaving or for display.

The drawer 340 contains a selection of ribbons of different colors and textures, each slightly wider than the width of the frame. The user might also include other materials such as, for example, feathers, yarn, string, and clip-on ornaments.

The user will thread ribbons in and out of the strings to create horizontal lines. This activity is fun for all ages, engages the creative mind, is tactile, and dementia-friendly. These form the horizontal weaving members 335, 325, 315 and 305 and vertical weaving members 310, 320 and 330.

Benefits of weaving:
a. Practices hand-eye coordination and concentration
b. Assists in retaining or developing fine motor skills in hands and fingers. Gripping, grasping, pinching, and holding.
c. Gives sensory stimulation
d. It is a quiet, mindful, calming activity
e. Helps develop creativity
f. Develops understanding of patterns and sequencing
g. The user can create new patterns each time
h. Functional decoration in the home
i. There is no right or wrong way to do this
j. Pride in accomplishment when finished a design
k. As a dementia activity or an activity for a child: all the above, plus the possibility of making it an engaging social activity by doing it one-on-one with another person and practicing language skills by naming colors and describing the process, i.e., in, out, etc.

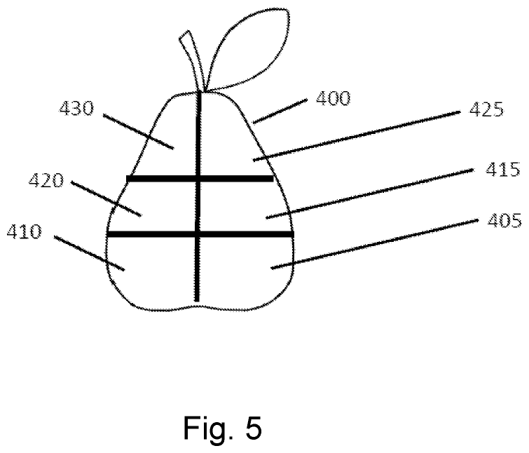

Referring to FIG. 5 is a Wood Puzzle 400 for people with dementia having puzzle pieces 405, 415, 425, 410, 420 and 430. This type of puzzle is targeted towards young children and adults with Alzheimer's/dementia, this puzzle activity promotes brain exercise such as problem-solving and decision-making, fine motor skills and visual perception. It encourages dexterity, social interaction, and positive emotions when completing a puzzle. The puzzle images are well-defined, colorful, and chosen with a view to add reminiscing to the activity. Images are specifically meant to induce reminiscence (i.e., favorite foods, older inventions, scenic, animals, etc.).
Puzzle piece size: from 2-8 inches across and 3-8 inches deep. Chunky to make them easy to move for hands that may have difficulty gripping things.
Puzzles are available in four or six parts.
An excellent activity for nursing homes, assisted living communities, and senior day centers, it can be used by individuals and one-on-one. Also, at-home professional and informal caregivers at home with the person they are caring for.
Suitable for children aged 3 to 6.

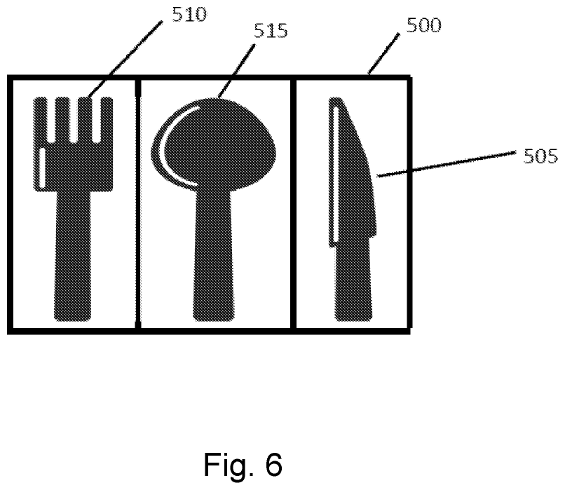

Referring to FIG. 6 is a Table Settings Puzzle 500 for people with dementia with puzzle pieces 505, 510 and 515. Many people with Alzheimer's/dementia have poor appetite or forget that it is mealtime. An activity that reminds them of food and eating, undertaken before meals, can trigger a feeling of hunger and encourage eating.

This puzzle activity encourages problem solving, decision-making, creative and convergent thinking, fine motor skills, visual perception, and gives the brain a workout. The components are easy to grip for fingers that may be lacking dexterity.

The jigsaw comprises a baseboard with a raised painted/varnished outside edge, 1″ wide and 0.2″ high to give the impression of a frame.

Included are 20-24 rectangular puzzle pieces, 2″×6″ and 0.4″ high. These rectangles all have a tablecloth printed background. The puzzle pieces have added printed elements. For example, a fork, knife, plate, spoon, napkin, salt, pepper, and food items such as cornflakes, pancakes, etc.

A puzzle piece can have one whole image, like a fork, and larger images need two or three pieces to complete the item, like a plate or a teacup.

You can fit twelve puzzle pieces within the raised edges of the baseboard in two rows.

The fact that there are more pieces than will fit on the board will engage the person to make choices. They could be asked to ‘lay a table setting’ for breakfast or dinner. They might be asked to lay a setting for the person across the table and have to do it upside-down, making it a more difficult activity. Individual pieces are higher than the outside edge of the puzzle, enabling the person to lift a piece easily if they want to rearrange the picture. This is a very flexible activity. For dementia patients with lower cognitive ability, you can use as few as two puzzle pieces to make, for example, a cup. You don't need the baseboard for this.

This is an excellent activity for nursing homes, assisted living communities, and senior day centers, it can be used by individuals, one-on-one, and small groups.

This activity has the potential to add more puzzle pieces of foods, etc.

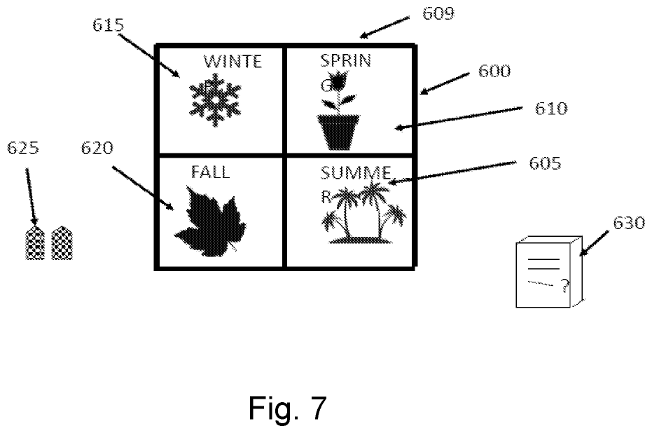

Referring to FIG. 7 which shows a Seasons, Reminiscing target game 600 for people with dementia. This printed, wipe-cleanable canvas floor mat 609 is preferably five feet square with one section for each of the four seasons. However, it can be any suitable size for the client or organization. Each section has four images relevant to that season image 610, image 605, image 620 and image 615. The mat will roll up for storage.

The game can be played from a seated or standing position.

The game comes with six colorful mini beanbags 625 for throwing, and a pack of cards 630 with simple questions related to the different seasons and particular images. When a participant answers the question, they must try to throw the beanbag 625 to the correct square.

With dementia, it is not all about competition. This game will encourage chat and reminiscing, participants are encouraged to throw to targets but are given several opportunities to succeed. Depending on the agility of the participants the floor mat can be placed near or further away.

The beanbags 625 are tactile and will exercise hands. Holding a beanbag 625 usually leads to automatic movement and massaging of the bag.

The throwing of the beanbag 625 exercises hand-eye coordination, fine motor skills, aim and accuracy, focus and concentration, movement, and fun.

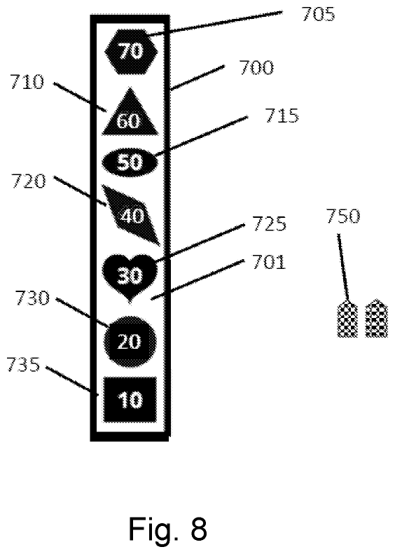

Referring to FIG. 8 is a shape, color, and numbers floor target game 700 for people with dementia. This target game is preferably three yards long and 18 inches wide. However, it can be any suitable size for the client or organization. It can also be produced in a square format. It is printed in vibrant colors and clear font numbers, and the canvas is cleanable. The mat 701 will roll up for storage.

The game comes with six colorful mini beanbags 750. People can challenge themselves to see how far they can throw to the different targets 705, 710, 715, 720, 725, 730 and 735 with the mat 701 placed so that the largest number is furthest away from them. They can aim to set personal bests in getting the far targets, be asked to throw on a specific color, number, shape, color of number etc.

This is an excellent game for dementia as it repeats and reinforces the memory of words/vocabulary for shapes, numbers, and colors.

For example, a caregiver can ask the participant to throw a bag 750 on a red target, or a triangle, a specific number, or a black or white number.

The target game 700 can be played seated or standing and the throwing of the beanbag exercises hand-eye coordination, fine motor skills, aim and accuracy, focus and concentration, movement, and fun. If played with others it has a social function.

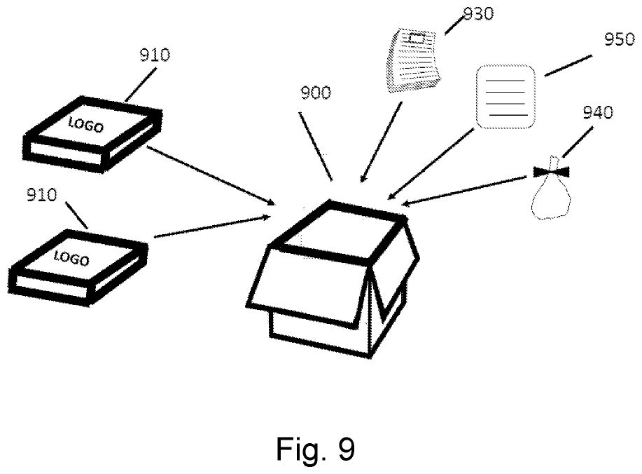

As shown in FIG. 9 an activity box can alternatively be provided for a specific organization and their activity. The contents of the activity box can be anything that the organization wants delivered to the user and it can have but not limited to items 910 with the organization logo, activity booklet 930, welcome information 950 and gift items 940. The items 910 with the organization logo, activity booklet 930, welcome information 950 and gift items 940 are placed in box 900 for delivery to the user. The box 900 can also have the organization logo on it.

In some embodiments the method or methods described above may be executed or carried out by a computing system including a tangible computer-readable storage medium, also described herein as a storage machine, that holds machine-readable instructions executable by a logic machine (i.e., a processor or programmable control device) to provide, implement, perform, and/or enact the above-described methods, processes and/or tasks. When such methods and processes are implemented, the state of the storage machine may be changed to hold different data. For example, the storage machine may include memory devices such as various hard disk drives, CD, or DVD devices. The logic machine may execute machine-readable instructions via one or more physical information and/or logic processing devices. For example, the logic machine may be configured to execute instructions to perform tasks for a computer program. The logic machine may include one or more processors to execute the machine-readable instructions. The computing system may include a display subsystem to display a graphical user interface (GUI) or any visual element of the methods or processes described above. For example, the display subsystem, storage machine, and logic machine may be integrated such that the above method may be executed while visual elements of the disclosed system and/or method are displayed on a display screen for user consumption. The computing system may include an input subsystem that receives user input. The input subsystem may be configured to connect to and receive input from devices such as a mouse, keyboard or gaming controller. For example, a user input may indicate a request that certain task is to be executed by the computing system, such as requesting the computing system to display any of the above-described information or requesting that the user input updates or modifies existing stored information for processing. A communication subsystem may allow the methods described above to be executed or provided over a computer network. For example, the communication subsystem may be configured to enable the computing system to communicate with a plurality of personal computing devices. The communication subsystem may include wired and/or wireless communication devices to facilitate networked communication. The described methods or processes may be executed, provided, or implemented for a user or one or more computing devices via a computer-program product such as via an application programming interface (API).

Since many modifications, variations, and changes in detail can be made to the described embodiments of the invention, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of the invention should be determined by the appended claims and their legal equivalents.

In addition, the present invention has been described with reference to embodiments, it should be noted and understood that various modifications and variations can be crafted by those skilled in the art without departing from the scope and spirit of the invention. Accordingly, the foregoing disclosure should be interpreted as illustrative only and is not to be interpreted in a limiting sense. Further it is intended that any other embodiments of the present invention that result from any changes in application or method of use or operation, method of manufacture, shape, size, or materials which are not specified within the detailed written description or illustrations contained herein are considered within the scope of the present invention.

Insofar as the description above and the accompanying drawings disclose any additional subject matter that is not within the scope of the claims below, the inventions are not dedicated to the public and the right to file one or more applications to claim such additional inventions is reserved.

Although very narrow claims are presented herein, it should be recognized that the scope of this invention is much broader than presented by the claim. It is intended that broader claims will be submitted in an application that claims the benefit of priority from this application.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. An interactive game kit for use by a person with dementia comprising:
a. a smart device;
b. a cloud application on a cloud server;
c. an application on said smart device;
d. an activity box;
e. said activity box containing at least one game;
f. at least one player having dementia wherein said at least one player having dementia receives said activity box and said at least one player having dementia using said application on said smart device access a website and said website communicating with said application on said smart device and said application on said smart device verifies said smart device is a sanction device using the IP address of said smart device and if the IP address is on a sanction IP list on said cloud application on a cloud server then said application on said smart device is allowed access to said website; and
g. said website communicates with said application on said smart device and displays instructions for said at least one game.

2. The interactive game kit of claim 1 wherein said smart device is selected from the group consisting of a computer, a smart device, a smart tablet and a smart phone;

3. The interactive game kit of claim 1 wherein said at least one game s selected from the group consisting of an all-in-one weaving kit, a wood puzzle for dementia, a table settings puzzle for dementia, a season, reminiscing target game for dementia, and a shape, color, and numbers floor target game for dementia;

4. A kit comprised of an activity box for use by a person with dementia comprising:
a. said activity box containing at least one game; and
b. said activity box containing an instruction sheet having instructions for said at least one game in said activity box.

5. The kit of claim 4 wherein said at least one game selected from the group consisting of an all-in-one weaving kit, a wood puzzle for dementia, a table settings puzzle for dementia, a season, reminiscing target game for dementia, and a shape, color, and numbers floor target game for dementia;
